(19) United States
(12) Patent Application Publication
(10) Pub. No.: US 2018/0028498 A1
(43) Pub. Date: Feb. 1, 2018

(54) A PHARMACEUTICAL COMPOSITION FOR THE PARENTERAL ADMINISTRATION OF MELATONIN, AND A PROCESS FOR ITS PREPARATION

(71) Applicant: INDUSTRIA FARMACEUTICA GALENICA SENESE S.R.L., Monteroni d'Arbia (Siena) (IT)

(72) Inventors: Danila Antonia PERILLO, Impruneta (Firenze) (IT); Giuseppe BUONOCORE, Siena (IT); Walter BALDUINI, Urbino (Pesaro-Urbino) (IT)

(21) Appl. No.: 15/552,984

(22) PCT Filed: Mar. 4, 2016
(86) PCT No.: PCT/IB2016/051235

§ 371 Date: Aug. 23, 2017

(30) Foreign Application Priority Data
Mar. 4, 2015 (IT) FI2015A000059

(51) Int. Cl. A61K 31/4045; A61K 9/00; A61K 47/02

(52) U.S. Cl. CPC A61K 31/4045; A61K 47/02; A61K 9/0019

(57) ABSTRACT

The present invention relates to pharmaceutical compositions for the parenteral administration of melatonin in the form of sterile aqueous solutions, provided with good stability even if devoid of any stabilising excipients and having a concentration of melatonin up to high amounts, sufficient for an efficacious medical treatment.

### FIELD OF THE INVENTION

The present invention relates in general to the pharmaceutical field, and more precisely it relates to a pharmaceutical composition for the parenteral administration of melatonin, and to the related process of preparation. The present composition does not contain any excipients, co-solvents and/or diluents different from the physiological saline solution.

### STATE OF THE ART

In the pharmaceutical field several examples are known of active principles that, once dissolved into a solution to be for instance injected, have instability problems. Often these problems come from the fact that the active principle easily oxidise and, after reaction with atmospheric oxygen and/or with the oxygen dissolved in the solution, generates unwanted degradation products that, practically, prevent the pharmaceutical use of the formulation.

One of these active principles is melatonin. Melatonin, whose chemical name is N-acetyl-5-methoxytriptamine, is a natural substance produced in humans by a gland, named pineal gland or epiphysis, placed at the base of the brain. This substance acts on the hypothalamus, a structure of the central nervous system that, among other functions, also controls the sleep function and, just by means of substances like melatonin, regulates the sleep-wake cycle. Among the first applications proposed for the synthetic melatonin produced in laboratory there was therefore, several decades ago, the application as a drug for the treatment of insomnia and, more in general, for the regulation of sleep. The pharmaceutical forms proposed for these applications were those traditional forms already used for oral administration, such as capsules and tablets, wherein melatonin was administered alone or in association with several other pharmaceutically active ingredients and with the typical excipients of these pharmaceutical forms. Over the years, many other therapeutic applications of melatonin have been proposed, for instance in the treatment of Parkinson's disease, depression, osteoporosis, migraine, and even in the treatment of tumour forms, generally including also in these cases the administration of melatonin always by the oral route.

There are moreover disclosed in the literature injectable formulations of melatonin, that are in general solutions of melatonin in ethanol or in propylene glycol, then diluted with water. As a matter of fact, melatonin is known to be a substance that dissolves in aqueous media with great difficulties. To dissolve melatonin, therefore, this active principle is generally dissolved in the form of powder in ethanol, where melatonin has a fairly good solubility, or in propylene glycol or in dimethyl sulphoxide, then the obtained solution can be diluted with water.

It is however reported in the literature how unstable are the ethanolic solutions of melatonin: already after two weeks from the preparation of the ethanolic solution, the thin layer chromatographic analysis shows the formation of at least further two components in the solution besides melatonin, clearly coming from the degradation of the product in solution. It is well known that, in order to render stable the pharmaceutical formulations showing this kind of problems of degradation, specific additives are used, just called as stabilisers. Obviously, these additives must be added in very low concentrations as they are added into pharmaceutical formulations, in particular if their administration route is the injection or infusion route, and they must be such as not to cause any kind of pharmacological or toxic effects, nor to interact in any way with the active principle and with the other excipients possibly present in the formulation. The stabilizing additives are generally anti-oxidants, such as ascorbic acid or derivatives thereof, or sulphites, having a protective function of the molecule of active principle from the action of light, of air and of other oxidants. The use of such additives is however not allowed in all types of formulations. In the formulations intended for paediatric patients, for instance, most of these excipients is not allowed, while there is a total ban on using any type of excipient or diluent other than the physiological saline solution in the case of formulations for injection or infusion to be administered to neonatal patients.

In the specific case of melatonin that, as said above, has a solubility in water close to zero and, when solubilized in solvents as ethanol, shows problems of stability due to oxidative degradation reactions, it seems completely impracticable its use as active principle in formulations intended for paediatric patients and a fortiori in formulations for neonatal patients.

Up to today some trials have been made to prepare parenteral formulations of melatonin, but all having an extremely low concentration of the active principle where only an aqueous medium is used, thus obtaining formulations not useful to treat any pathologies. In other cases excipients and/or diluents are used, which are not allowed in formulations intended for paediatric or neonatal patients.

An example of this latter type of formulation was described in the International patent application published under No. WO 2012/156565, just directed to an injectable formulation of melatonin, wherein the water solubilisation of the active principle is obtained thanks to the addition of propylene glycol, an excipient commonly used to increase the solubility in water of active principles scarcely soluble in water. The amount of propylene glycol added to the formulation is very high: in the only example of preparation included in WO 2012/156565 the amount of glycol is equal to 25% by volume with respect to the total volume of the solution. And in fact the target of patients for these formulations seems to be mainly that of adult patients, for the treatment of various pathologies, therefore without any limitation on the use of excipients and co-solvents, even if a possible use of these formulations in the treatment of neonatal sepsis was mentioned too. This latter was clearly just a suggestion not having any actual application in the medical practice, where any type of excipient and/or diluent other than the saline solution is categorically prohibited for the treatments with melatonin of neonatal patients. Furthermore, the experimental stability tests described for these formulations seem not to be reliable: the tests are in fact carried out for a first period of 3 months after the preparation, without investigating the long term stability, and above all these tests are limited to a simple observation of the appearance of the solution and to the evaluation of the melatonin concentration obtained by HPLC analysis according to a protocol not disclosed nor referred to in any acknowledged pharmacopeia. The results of the tests, even if showing a certain variability of concentration of the active principle, are said as proving the stability of the tested formulations; in particular, according to what reported in WO 2012/156565, a formulation of melatonin in water with 25% propylene glycol, diluted in physiological solution, is considered as stable for 7 days starting from the preparation, with a variability of the melatonin concentration ranging between 10 and 13%. Along with these little comforting data, also the control of bacterial endotoxins present in the formulation, which are of about 5 EU/ml, does not encourage a possible use of these formulations for the treatment of neonatal patients.

As far as the Applicant is aware of, also in view of what said above, up to today pharmaceutical compositions for the parenteral administration of melatonin that resolve the problems highlighted above for the known compositions have not been prepared. In other words, in particular, up to now, parenteral formulations of melatonin are not available, that are sufficiently stable over time and are completely without excipients, co-solvents or diluents different from the physiological saline solution, but containing a sufficient amount of active principle for an efficacious medical treatment.

### SUMMARY OF THE INVENTION

Now the Applicant has found a process of preparation to yield, by a procedure that can be scaled up also at industrial level, pharmaceutical compositions suitable for the parenteral administration of melatonin. These compositions overcome the above mentioned problems of the known pharmaceutical compositions, since these novel compositions are stable and sterile, and at the same time they provide a sufficient amount of active principle to achieve an efficacious medical treatment. Thanks to these characteristics, such novel compositions are suitable for the use in the treatment of paediatric patients or neonatal patients.

It is therefore a subject of the invention a pharmaceutical composition for the parenteral administration of melatonin in the form of a stable aqueous solution, as defined in the first of the attached claims.

This pharmaceutical composition for the use in the treatment of hypoxic-ischemic encephalopathy in neonatal patients, and the process of preparation of this pharmaceutical composition, as defined in the independent claims 7 and 9, are further subject of the invention.

Further important characteristics of the pharmaceutical compositions, of the related process of preparation, and of their use according to the invention are defined in the dependent claims here attached, and reported in the following detailed description.

### DETAILED DESCRIPTION OF THE INVENTION

A pharmaceutical composition was found that is stable and sterile for the parenteral administration of melatonin in the form of an aqueous solution, comprising melatonin and physiological saline solution, having a concentration of melatonin ranging between 0.2 and 0.4 mg/ml and without any excipients, co-solvents and/or diluents different from the physiological saline solution.

In the present invention by “physiological saline solution” or “saline solution” is meant a sodium chloride solution in purified water or, preferably, in water for injectable preparations (in the following abbreviated by “water for injections”). The water for injection is far the most used medium for preparing injectable formulations of drugs, and it is obtained by distillation of purified water, or of at least drinking water, in a distiller having all the parts intended for contacting water made of quartz or of metal steel, or again having these parts made anyway not attackable by means of a suitable coating. The water distilled under these conditions is sterile and not pyrogen, i.e. it is free from germs, from microorganisms, and from any elements that may cause fever, named “pyrogen”; it is packed so as to remain in these conditions until use for the preparation of the injectable pharmaceutical composition. To this aim are generally used collecting containers previously washed with water for injections, filled for almost all the volume, hermetically closed so as to avoid any bacterial contaminations from the outside and maintained at such a temperature that the development of microorganisms inside the container is not promoted.

According to a preferred embodiment of the invention, the physiological saline solution used in the preparation of the present pharmaceutical compositions has a concentration equal to 0.9% by weight of sodium chloride with respect to the total volume of the solution; this concentration is indeed that typical of the commonly used physiological saline solution, and therefore much more available and ready to be used.

The present pharmaceutical compositions in the form of an aqueous solution, thanks to the process of preparation of the invention described in detail in the following, may contain, dissolved in aqueous solution, amounts of melatonin that are relatively high and suitable for the dosages required in medical treatments; the concentration of melatonin in the present compositions is indeed comprised between 0.2 and 0.4 mg/ml, this latter being the maximum value of concentration obtained for the melatonin in the present aqueous compositions, in the absence of any excipients, co-solvents or diluents different from the physiological saline solution.

The melatonin used for preparing the compositions of the invention may be for instance synthetic melatonin in powder of injectable pharmaceutical grade, or it may be used as starting compound a derivative, a salt, a solvate or a prodrug of melatonin.

The pharmaceutical compositions subject of the present invention have been prepared by a process of preparation comprising dissolving the melatonin in powder in a physiological saline solution, defined as said above, in a suitable pharmaceutical dissolver, wherein the dissolver, the physiological saline solution and the melatonin solution obtained are deaerated with an inert gas, preferably with filtered nitrogen.

The melatonin dissolution is preferably carried out at temperature ranging between 50 and 60° C., in a physiological saline solution freshly prepared by addition of NaCl in water for injections, directly in the pharmaceutical dissolver.

The content of oxygen dissolved in solution is constantly monitored at the aim of maintaining it at values lower than 2 ppm. Also the processing of the solution subject of the invention after the solution's preparation, including the bottling, or anyway the packaging in containers of the desired dosage, are carried out under inert gas, preferably under filtered nitrogen, controlling again the content of oxygen dissolved in the solution.

According to a preferred embodiment of the invention, the present pharmaceutical compositions of melatonin in physiological saline solution are packed in containers made of Type I glass, i.e. of glass of amber colour, suitable for the preparations of products sensitive to ultraviolet rays. Containers of Type I glass suitable for packing and storing the present compositions are for instance vials, in particular 10 ml vials. Obviously these containers have to guarantee maintenance of the desired conditions of deaeration, wherein the content of O₂ is always lower than 2 ppm; so, for instance, the vials, once they are filled up under inert gas atmosphere, are closed by torch welding of the molten glass and checked with an apparatus of the type Leaker Test to test the seal of the vial.

The present pharmaceutical compositions are sterilisable, in particular they are autoclavable, for example by treatment in a super-heated water autoclave. A sterilisation cycle commonly used for injectable products in glass vials may be used, at 118° C. with F0=15 (the exposition of the product is equivalent to a temperature of 121° C. for 15 minutes, valid for an absolute sterilisation).

The specific conditions of the process of preparation and the use of deaerated physiological solution only, that maintain the pH values of the compositions between 5 and 7, cause the present pharmaceutical compositions to be characterised by a good stability over time despite the high concentration of the active principle.

The pharmaceutical compositions according to the present invention are suitable for the parenteral administration of melatonin by any one of the known parenteral administration routes, such as the injection or infusion route, that may be intramuscular, intravenous, intradermal, subcutaneous, intra-arterial, or intrathecal. A preferred administration route is the intravenous infusion.

For the above said reasons, and in particular for the complete absence of any excipients, co-solvents or diluents different from the physiological saline solution, the present pharmaceutical compositions are suitable for the administration to paediatric patients or also mostly to neonatal patients.

The melatonin may find a therapeutic application in the treatment of hypoxic-ischemic encephalopathy in infants, and therefore the present pharmaceutical compositions are particularly useful in the treatment of this serious disease. The hypoxic-ischemic encephalopathy is indeed one of the major reasons of neonatal death and neurological disability in children. The estimated incidence of this disease is of about 1-2/1000 infants born at term and increases up to 60% among premature infants weighing less than 1500 grams. A percentage comprised between 20 and 50% of asphyxiated infants who develop a hypoxic-ischemic encephalopathy unfortunately are destined to die in the neonatal period, while about 25% of the survivors have severe neurological disabilities such as cerebral palsy, mental retardation, epilepsy, and learning disorders. The present pharmaceutical compositions, for the absence of any excipients, co-solvents or diluents different from the physiological saline solution, for the dosage of melatonin and for the pharmaceutical form suitable for the parenteral administration, are particularly appropriate for the treatment of this serious pathology in infants.

Melatonin is a natural hormone physiologically secreted in humans by the pineal gland with a circadian rhythm; however, the human foetus is not able to produce melatonin, but receives it only from the mother through placenta. And this is the reason why in premature infants a deficiency of this substance, essential for normal neurodevelopment, is observed (Merchant NM. et al. Br. J. Clin. Pharmacol. 2013 November; 76(5): 725-33).

Besides the treatment of the hypoxic-ischemic encephalopathy, the melatonin proved to be efficacious in other types of neonatal diseases. In a study, for example, infants have been considered having bronchopulmonary dysplasia and subjected to ventilation. This kind of treatment causes a lung injury, associated to the formation of free radicals (pro-inflammatory cytokines). Melatonin has been shown in this case effective in reducing the production of these molecules thanks to its antioxidant action (Gitto E. et al, Journal of Pineal Research 2005 October; 39(3):287-293).

The same efficacy has been demonstrated in infants with chronic lung disease (Gitto E. et al, Journal of Pineal Research 2004 May; 36(4):250-5).

Infants, compared to children and adults, are more sensitive to pain. Often, in the intensive care units, premature infants are subjected to treatments such as endotracheal intubation, an extremely painful procedure. Also in these cases it is known the involvement of cytokines as molecules responsible for the induction and for the perception of pain.

A study demonstrated the efficacy of melatonin as an analgesic, in combination with other drugs, during endotracheal intubation in infants (Gitto E. et al, Journal of Pineal Research 2012 April; 52(3):291-5).

The following non limiting examples are reported to illustrate the present invention.

### Example 1

Preparation of the Pharmaceutical Composition

A pharmaceutical dissolver of suitable size was deaerated by insufflation of filtered nitrogen, then the dissolver was charged with water for injections at 85° C., to fill approximately 80% of the inner volume of the dissolver. After cooling water at 50° C., sodium chloride was added in such amount as to obtain a final concentration of 9mg/ml; the so obtained solution was deaerated by bubbling filtered nitrogen. It was then added melatonin in powder in amount of 0.4 mg/ml, by mixing until dissolution, and the temperature was lowered to about 30° C., then adding water for injections up to the final volume.

The mixture was then stirred until an homogeneous solution was obtained, moreover bubbling filtered nitrogen up to a volumetric content of O₂ lower than 2 ppm.

The so obtained solution was analysed to check the concentration of melatonin and of sodium chloride, the pH, the density, the absence of bacteria by bioburden, thus finding that the solution meets the desired characteristics described above. In particular the level of bacterial endotoxins was checked by LAL test, obtaining a value lower than the threshold of 0.25 EU/ml. The concentration of melatonin in solution was evaluated by HPLC.

In order to identify possible impurities in the solution prepared as described above the following “stress tests” have been carried out: photo-degradation carried out by exposure of the samples to UV for 3 hours; thermal hydrolysis at 60° C. with analysis of the samples at time t₀, after 3 days and after 10 days; acid and basic hydrolysis in HCl 1N and in NaOH 1N, respectively, maintaining the samples at 25° C. with analysis at time t₀, after 3 days and after 10 days; oxidative stress by exposure to 3% hydrogen peroxide for 3 hours.

These studies revealed the formation of the degradation products 6-hydroxymelatonin (6-OHM) and N1-acetyl-N2-formyl-5-methoxykynuramine (AFMK), that are endogenous hepatic metabolites, therefore non-toxic, and provided with antioxidant activity. These products only form in specific conditions: in particular, AFMK forms following a treatment with 3% hydrogen peroxide for 3 hours and by addition of NaOH 1 N after 3 days; 6-OHM forms following a treatment with NaOH 1 N after 3 days. In all the other conditions of stress test described above the formation of the above said degradation products or of other impurities was not observed.

The melatonin solution obtained as described above was then subjected to stability tests, both accelerated and normal, without detecting any variations of the concentration of active principle in the short-medium term. The tests carried out included in particular the storage of a portion of the solution at 40° C. with 75% of relative humidity for 6 months with controls every 30 days (accelerated stability test), and the storage of another portion at room temperature of about 25° C., with 40% of relative humidity for 24 months in total and controls every month for the first 6 months and every 6 months in the subsequent period (stability test is ongoing).

Furthermore, the samples subject of the stability tests are completely free from the above mentioned impurities.

### Example 2

Treatment of the Solution and Packaging

The aqueous solution of melatonin obtained as described above in Example 1 was filtered and divided in 10 ml single-dose vial as follows.

A suitable transfer line, a filtration plant and a break tank connected to a vials filling machine were deaerated by insufflation of filtered nitrogen, up to an internal pressure of O₂ lower than 2 ppm, while the vials filling machine was also arranged to work under filtered nitrogen atmosphere, by analysing “in process” for checking the concentration of sodium chloride and melatonin divided by vial. 10 ml vials, made of

Type I amber glass, have been used. Start and end of the batch for the analysis of the final product have been marked, by taking an appropriate starting sample to check bacterial endotoxins by the bioburden test and to analyse concentration, pH and volumetric content of O₂, before the sterilisation, according to the procedures described above in details, which confirmed the results already verified above for the solution. During the filling, the volume of the distributed solution was moreover periodically checked, to confirm homogeneity of the dosage.

The filled vials were then subjected to sterilisation in perforated baskets placed in a super-heated water autoclave with a cycle set up for 10 ml glass vials at 118° C. with F0=15.

After sterilisation, the vials were passed individually to an automatic plant for a Leaker test, which guarantees the perfect welding of the tip of the vials and their integrity; finally, the welded vials were passed to an automatic plant for optical inspection in order to verify the presence of visible particles and to check the level of solution in each vial, so that any possible defective vial may be discarded.

1. A stable and sterile pharmaceutical composition for the parenteral administration of melatonin in the form of an aqueous solution, comprising melatonin and physiological saline solution, said pharmaceutical composition having a melatonin concentration of between 0.2 and 0.4 mg/ml and being completely free of any excipients, co-solvents and/or diluents different from said physiological saline solution.

2. The pharmaceutical composition according to claim 1, consisting essentially of melatonin, purified water and sodium chloride.

3. The pharmaceutical composition according to claim 2, wherein said sodium chloride concentration is equal to 0.9% by weight with respect to the total volume of the solution.

4. The pharmaceutical composition according to claim 2, consisting of a solution of melatonin in concentration equal to 0.4 mg/ml in a physiological solution of sodium chloride in purified water with a concentration of sodium chloride equal to 0.9% by weight with respect to the total volume of the solution.

5. The pharmaceutical composition according to claim 1, which is packaged in Type I glass containers.

6. The pharmaceutical composition according to claim 1, which is sterilizable by autoclaving.

7. A method for the treatment of hypoxic-ischemic encephalopathy in neonatal patients, comprising administering a composition of claim 1 to a neonatal patient in need thereof.

8. The method according to claim 7, wherein said composition is administered by intravenous infusion.

9. A process for the preparation of a pharmaceutical composition in the form of an aqueous solution of melatonin as defined in claim 1, comprising dissolving melatonin in powder in a physiological saline solution in a pharmaceutical dissolver, wherein said dissolver, said physiological saline solution and said melatonin solution are deaerated by injecting an inert gas so as to maintain the volumetric content of residual O₂ at values lower than 2 ppm.

10. The process according to claim 9, further comprising packaging said aqueous solution of melatonin in single doses under atmosphere of an inert gas.

11. The process according to claim 9, wherein said inert gas is nitrogen previously subjected to filtration.
